(19) United States
(12) Patent Application Publication
(10) Pub. No.: US 2023/0233255 A1
(43) Pub. Date: Jul. 27, 2023

(54) MEDICAL DEVICE AND SHUNT FORMING METHOD

(71) Applicant: TERUMO KABUSHIKI KAISHA, Tokyo (JP)

(72) Inventor: Yusuke TAKAHASHI, Hadano-shi (JP)

(73) Assignee: TERUMO KABUSHIKI KAISHA, Tokyo (JP)

(21) Appl. No.: 18/192,412

(22) Filed: Mar. 29, 2023

Related U.S. Application Data
(63) Continuation of application No. PCT/JP2021/035233, filed on Sep. 27, 2021.

(30) Foreign Application Priority Data
Sep. 30, 2020 (JP) 2020-164557

(51) Int. Cl. A61B 18/14

(52) U.S. Cl. CPC A61B 18/1492; A61B 2017/00247

(57) ABSTRACT

An expansion body that radially expands and contracts; an elongated shaft portion having a distal portion including a proximal end fixing portion to which a proximal end of the expansion body is fixed; a plurality of electrode portions provided along the expansion body are included, and the expansion body includes a recess portion recessed radially inward when the expansion body expands and defining a reception space that receives a biological tissue. The recess portion includes a bottom portion positioned at a radial innermost side, a proximal side upright portion extending radially outward from a proximal end of the bottom portion, and a distal side upright portion extending radially outward from a distal end of the bottom portion. One of two electrode portions that are circumferentially adjacent to each other is arranged at the proximal side upright portion, and the other is arranged at the distal side upright portion.

### CROSS-REFERENCES TO RELATED APPLICATIONS

This application is a continuation of International Patent Application No. PCT/JP2021/035233 filed on Sep. 27, 2021, which claims priority to Japanese Patent Application No. 2020-164557 filed on Sep. 30, 2020, the entire content of both of which is incorporated herein by reference.

### TECHNOLOGICAL FIELD

The present invention generally relates to a medical device and a shunt forming method that impart energy to a biological tissue.

### BACKGROUND DISCUSSION

As a medical device, there is known one in which an electrode portion is disposed on an expansion body that expands and contracts in a biological body, and treatment is performed by ablation, which involves cauterizing a biological tissue by a high-frequency current from the electrode portion. As a treatment by ablation, the shunt treatment on the atrial septum is known. For patients who suffer from heart failure, by forming, in an atrial septum, a shunt (puncture hole) serving as an escape route for increased atrial pressure, the shunt treatment enables heart failure symptoms to be alleviated. In the shunt treatment, the atrial septum is accessed using an intravenous approaching method, and the puncture hole is formed to have a desired size. Such a medical device is disclosed in, for example, International Patent Application Publication No. 2019-85841 (WO 2019-85841).

### SUMMARY

In a medical device that forms a shunt in an atrial septum, an electrode portion is arranged to come into contact with only one of both sides of the atrial septum. In this case, since the energy of cauterization is imparted only from one side of the atrial septum, there is a possibility that sufficient cauterization cannot be performed depending on the thickness of the septal tissue. In addition, when energy is imparted for a long time in order to obtain sufficient cauterization, a site locally raised to a high temperature is generated between the electrode portions, so that the risk of formation of a thrombus increases.

A medical device and a shunt forming method disclosed here are configured to evenly cauterize a biological tissue in a thickness direction and suppress the biological tissue to be cauterized from being locally raised to a high temperature.

In view of the above, a medical device includes: an expansion body configured to expand and contract in a radial direction; an elongated shaft portion having a distal portion including a proximal end fixing portion to which a proximal end of the expansion body is fixed; a plurality of electrode portions provided along the expansion body, in which the expansion body includes a recess portion recessed radially inward when the expansion body expands and defining a reception space configured to receive a biological tissue, the recess portion includes a bottom portion positioned on a radial innermost side, a proximal side upright portion extending radially outward from a proximal end of the bottom portion, and a distal side upright portion extending radially outward from a distal end of the bottom portion, and one of two electrode portions circumferentially adjacent to each other is arranged at the proximal side upright portion, and the other is arranged at the distal side upright portion.

A shunt forming method comprises forming a shunt in an atrial septum using a medical device including an expansion body configured to expand and contract in a radial direction, an elongated shaft portion having a distal portion including a proximal end fixing portion to which a proximal end of the expansion body is fixed, and an even number of electrode portions provided along the expansion body. The electrode portions includes first and second electrode portions that are circumferentially adjacent to each other. The expansion body also includes a recess portion defining a reception space, the recess portion including a bottom portion positioned at a radial innermost side, a proximal side upright portion extending radially outward from a proximal end of the bottom portion, and a distal side upright portion extending radially outward from a distal end of the bottom portion, with the first electrode portion being arranged at the proximal side upright portion and the second electrode portion being arranged at the distal side upright portion. The method comprises: positioning the recess portion in a puncture hole in atrial septum so that biological tissue surrounding the puncture hole is positioned in the reception space, and bringing the electrode portion into contact with the biological tissue that is positioned in the reception space, and applying voltage to the electrode portion arranged in the proximal side upright portion and the electrode portion arranged in the distal side upright portion to cauterize the biological tissue in the reception space.

In the medical device configured as described above, since the electrode portions are configured to be alternately brought into contact with both surfaces of the biological tissue received in the reception space of the recess portion, it is possible to reduce the region where the energy from the circumferentially adjacent electrode portions overlaps, evenly cauterize the biological tissue, and prevent the biological tissue from being raised to an excessively high temperature.

The expansion body may include a plurality of wire portions defining the recess portion to include four or more recess portions arranged at equal intervals in the circumferential direction of the expansion body, the plurality of recess portions may each include the bottom portion, the proximal side upright portion, and the distal side upright portion, and the electrode portions may be disposed in the plurality of recess portions in a one-to-one manner. This allows the electrode portion to more evenly cauterize the biological tissue.

An even number of the electrode portions may be provided in the circumferential direction. This allows all the electrode portions to be alternately arranged with respect to both surfaces of the biological tissue.

In the shunt forming method configured as described above, since the electrode portions are configured to be alternately brought into contact with both surfaces of the biological tissue, it is possible to reduce the region where the energy from the circumferentially adjacent electrode portions overlaps, evenly cauterize the biological tissue, and prevent the biological tissue from being raised to an excessively high temperature.

When the biological tissue is cauterized, a proximal side cauterization operation and a distal side cauterization operation may be alternately performed, the proximal side cauterization operation including cauterizing the biological tissue by applying a voltage to an electrode portion arranged in the proximal side upright portion of the electrode portion, and the distal side cauterization operation including cauterizing the biological tissue by applying a voltage to an electrode portion arranged in the distal side upright portion of the electrode portion. Due to this, the proximal side cauterization operation and the distal side cauterization operation are alternately performed, energy is imparted simultaneously from the electrode portions separated by two in the circumferential direction, and the distance between the electrode portions imparted with the voltage can be increased, and therefore the temperature rise of the biological tissue due to the cauterization can be further suppressed.

### BRIEF DESCRIPTION OF THE DRAWINGS

FIG. 1 is a front view showing an overall configuration of a medical device according to an embodiment.

FIG. 2 is an enlarged perspective view of the vicinity of an expansion body.

FIG. 3 is an enlarged front view of the vicinity of the expansion body.

FIG. 4A is a cross-sectional view taken along the section line 4A-4A in FIG. 3 and FIG. 4B is a cross-sectional view taken along the section line 4B-4B in FIG. 3.

FIG. 5A is a cross-sectional view obtained by developing, in the circumferential direction, a biological tissue with which electrode portions alternately arranged with respect to both surfaces of the biological tissue are in contact along the circumferential direction, and FIG. 5B is a cross-sectional view obtained by developing, in the circumferential direction, a biological tissue with which electrode portions arranged only on one surface side of the biological tissue are in contact along the circumferential direction.

FIG. 6 is a view showing an expansion body stored in a storage sheath.

FIG. 7 is a flowchart of a treatment method using a medical device.

FIG. 8 is a view for schematically describing a state where the expansion body is disposed in an atrial septum, in which the medical device is shown in a front view and the biological tissue is shown in a sectional view, respectively.

FIG. 9 is an enlarged view of the vicinity of the expansion body in FIG. 6.

FIG. 10 is a view for schematically describing a state where a diameter of the expansion body is increased in the atrial septum from the state of FIG. 7.

FIG. 11 is a flowchart of a treatment method including a voltage application method according to a modification example.

FIGS. 12A and 12B are circumferential developed cross-sectional views of a biological tissue in the voltage application method according to the modification example, in which FIG. 12A is a view showing a state in which a voltage is applied to electrode portions arranged in a proximal side upright portion and FIG. 12B is a state in which a voltage is applied to electrode portions arranged in a distal side upright portion.

FIG. 13 is an enlarged view of the vicinity of an expansion body according to a first modification example.

FIG. 14 is an enlarged view of the vicinity of an expansion body according to a second modification example.

FIGS. 15A and 15B are enlarged views of the vicinity of an expansion body according to a third modification example.

### DETAILED DESCRIPTION

Hereinafter, an embodiment of the medical device and shunt forming method will be described with reference to the drawings. In some cases, dimensional ratios in the drawings may be exaggerated and different from actual ratios for convenience of description. In addition, in the present specification, a side on which a medical device 10 is inserted into a biological lumen will be referred to as a “distal end” or a “distal side”, and an operating hand-side will be referred to as a “proximal end” or a “proximal side”.

The medical device 10 according to the embodiments described in this disclosure may be configured as follows. A puncture hole Hh formed in an atrial septum HA of a patient's heart H is enlarged, and further, a maintenance treatment is performed so that the puncture hole Hh having an increased diameter or size is maintained to have an increased size.

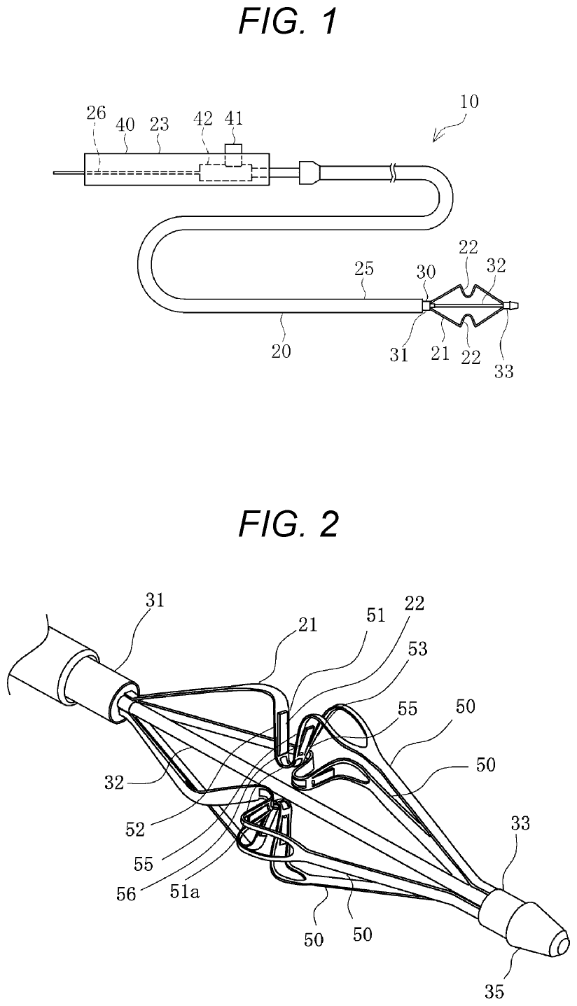

As shown in FIG. 1, the medical device 10 according to the present embodiment includes an elongated shaft portion 20, an expansion body 21 disposed in a distal portion of the shaft portion 20, and a hand operation unit 23 disposed in a proximal portion of the shaft portion 20. The expansion body 21 has an electrode portion or electrode 22, which is an energy transfer element for performing the above-described maintenance treatment. The expansion body 21 includes plural electrode portions or electrodes 22. The description below referring to an electrode portion or electrode is understood to apply to all of the electrode portions or electrodes 22.

The shaft portion 20 has a distal portion 30 including a proximal end fixing portion or part 31 to which the proximal end of the expansion body 21 is fixed and a distal end fixing portion or part 33 to which the distal end of the expansion body 21 is fixed. The distal portion 30 of the shaft portion 20 has a shaft extension portion 32 extending in the expansion body 21 from the proximal end fixing portion 31. The shaft portion 20 has a storage sheath 25 disposed at the outermost peripheral portion. The expansion body 21 is movable forward and rearward from (i.e., relative to) the storage sheath 25 in an axial direction. In a state where the storage sheath 25 is moved to the distal side of the shaft portion 20, the storage sheath 25 can internally store the expansion body 21. In a state where the expansion body 21 is stored in the storage sheath 25, the storage sheath 25 is moved to the proximal side. In this manner, the expansion body 21 can be exposed.

The shaft portion 20 includes a pulling shaft 26. The pulling shaft 26 is disposed from the proximal end of the shaft portion 20 to the shaft extension portion 32, and the distal portion is fixed to a distal member 35.

The distal portion of the pulling shaft 26 is fixed is fixed to the distal member 35. The distal member 35 may not be fixed to the expansion body 21. In this manner, the distal member 35 can pull the expansion body 21 in a contracting direction. In addition, when the expansion body 21 is stored in the storage sheath 25, the distal member 35 is separated to the distal side from the expansion body 21. Accordingly, the expansion body 21 can be rather easily moved in an axial direction, and storage capability can be improved.

The hand operation unit 23 has a housing 40 configured to be held by an operator, an operation dial 41 that can be rotationally operated by the operator, and a conversion mechanism 42 operated in conjunction with the rotation of the operation dial 41. The pulling shaft 26 is held inside the hand operation unit 23 by the conversion mechanism 42. In conjunction with the rotation of the operation dial 41, the conversion mechanism 42 can move the held pulling shaft 26 forward and backward along the axial direction. For example, a rack and pinion mechanism can be used as the conversion mechanism 42.

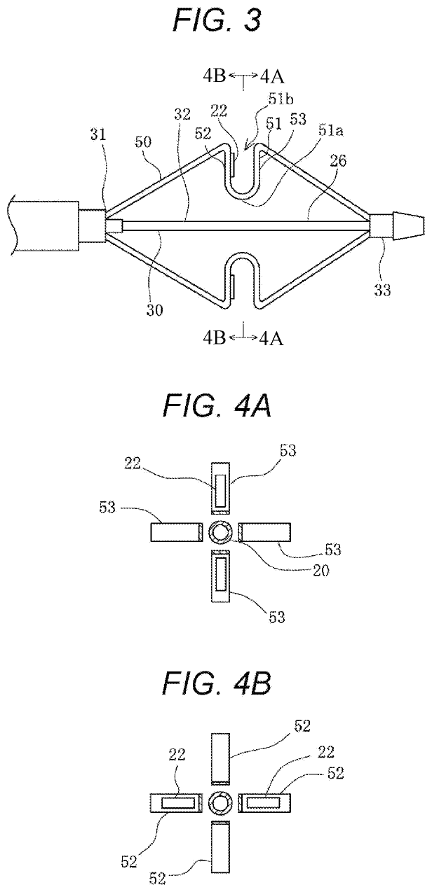

The expansion body 21 will be described in more detail. As shown in FIGS. 2 and 3, the expansion body 21 has a plurality of wire portions 50 in a circumferential direction. In the present embodiment, for example, four of the wire portions 50 are disposed in the circumferential direction. The wire portions 50 are respectively configured to expand and contract in a radial direction. A proximal portion of each wire portion 50 extends to a distal side from the proximal end fixing portion 31. A distal portion of each wire portion 50 extends to a proximal side from the distal end fixing portion 33. Each wire portion 50 is inclined to increase in the radial direction (i.e., to be spaced further radially outwardly) from both end portions toward a central portion in an axial direction. In addition, the central portion of each wire portion 50 in the axial direction has a recess portion or recess 51 recessed radially inward of the expansion body 21. A radially innermost portion of the recess portion 51 is a bottom portion 51a. The recess portion 51 defines a reception space 51b configured to receive a biological tissue when the expansion body 21 expands.

The recess portion 51 includes a proximal side upright portion 52 extending radially outward from the proximal end of the bottom portion 51a and a distal side upright portion 53 extending radially outward from the distal end of the bottom portion 51a. The electrode portion 22 is disposed on the proximal side upright portion 52 or the distal side upright portion 53 so as to face the reception space 51b. In the distal side upright portion 53, a central portion in a width direction has a slit shape. The distal side upright portion 53 has an outer edge portion 55 on both sides and a backrest portion 56 of the central portion.

For example, the wire portion 50 forming the expansion body 21 has a flat plate shape cut out from a cylinder. The wire forming the expansion body 21 can have, for example, a thickness of 50 μm to 500 μm and a width of 0.3 mm to 2.0 mm. However, the wire may have a dimension outside this range. In addition, the wire portion 50 may have a circular shape in a cross section, or may have other shapes in a cross section.

For example, the electrode portion 22 is configured to include a bipolar electrode that receives electric energy from an energy supply device (not illustrated) serving as an external device. In this case, electricity is supplied to the electrode portion 22 disposed in each of the wire portions 50. The electrode portion 22 and the energy supply device are connected to each other by a conducting wire (not illustrated) coated with an insulating coating material. The conducting wire is drawn outward (i.e., extends) via the shaft portion 20 and the hand operation unit 23, and is connected to the energy supply device.

Alternatively, the electrode portion 22 may be configured to serve as a monopolar electrode. In this case, the electricity is supplied from a counter electrode plate prepared outside a body. In addition, the electrode portion 22 may alternatively be a heating element (electrode chip) that generates heat by receiving high-frequency electric energy from the energy supply device. In this case, the electricity is supplied to the heating element disposed in each of the wire portions 50. Furthermore, the electrode portion 22 can be configured to include an energy transfer element that applies energy to the puncture hole Hh, such as a heater including an electric wire which provides heating and cooling operation or generating frictional heat by using microwave energy, ultrasound energy, coherent light such as laser, a heated fluid, a cooled fluid, or a chemical medium. A specific form of the energy transfer element is not particularly limited.

The wire portion 50 is configured to be formed of a metal material. For example, the metal material of the frame 70 can be a titanium-based (Ti—Ni, Ti—Pd, or Ti—Nb—Sn) alloy, a copper-based alloy, stainless steel, β-titanium steel, or a Co—Cr alloy. An alloy having a spring property such as a nickel titanium alloy may also be used as the material. However, a material of the wire portion 50 is not limited, and the frame 70 may be formed of other materials.

It is preferable that the shaft portion 20 is formed of a material having a certain degree of flexibility. For example, the materials of the shaft portion 20 may include polyolefin such as polyethylene, polypropylene, polybutene, ethylene-propylene copolymer, ethylene-vinyl acetate copolymer, ionomer, and a mixture of the above-described two or more materials, fluororesin such as soft polyvinyl chloride resin, polyamide, polyamide elastomer, polyester, polyester elastomer, polyurethane, and polytetrafluoroethylene, polyimide, PEEK, silicone rubber, or latex rubber.

For example, the pulling shaft 26 can be formed of the materials in which an elongated wire formed of a super elastic alloy such as a nickel-titanium alloy and a copper-zinc alloy, a metal material such as stainless steel, or a resin material having relatively high rigidity is coated with a resin material such as polyvinyl chloride, polyethylene, polypropylene, and ethylene-propylene copolymer.

For example, the distal member 35 can be formed of a polymer material such as polyolefin, polyvinyl chloride, polyamide, polyamide elastomer, polyurethane, polyurethane elastomer, polyimide, and fluororesin or a mixture of polymer materials. Alternatively, the distal member 35 can be formed of a multilayer tube containing two or more polymer materials.

As shown in FIGS. 4A and 4B, the electrode portions 22 circumferentially adjacent to each other are disposed in any of the proximal side upright portion 52 and the distal side upright portion 53 not in a continuous manner but in an alternate manner. That is, one of the two electrode portions 22 circumferentially adjacent to each other is disposed in the proximal side upright portion 52, and the other is disposed in the distal side upright portion 53. Thus, the electrode portions 22 are circumferentially shifted relative to one another so that they are located at different circumferential positions and there is no circumferential overlap between the electrode portions 22. In other words, all of the electrode portions 22 are located at different circumferential positions on the expansion body as shown by way of example in FIGS. 2, 4A, 4B and 5A. The four electrode portions 22 are all arranged at equal intervals in the circumferential direction of the expansion body 21. When the biological tissue is received in the reception space 51b of the recess portion 51, the electrode portion 22 arranged in the proximal side upright portion 52 comes into contact with one surface of the biological tissue, and the electrode portion 22 arranged in the distal side upright portion 53 comes into contact with the other surface of the biological tissue.

FIGS. 5A and 5B present, by hatching, a range in which the high-frequency energy from the electrode portion 22 propagates at a constant intensity. As shown in FIG. 5A, the two circumferentially adjacent electrode portions 22 are respectively brought into contact with one surface and the other surface of the biological tissue, so that it is possible to reduce an overlapping region in a range where the high-frequency energy reaches from each of the electrode portions 22. As shown in FIG. 5B, when all the electrode portions 22 are brought into contact with the same surface of the biological tissue, ranges where the high-frequency energy from the adjacent electrode portions 22 reaches overlap in a partial region X. In this region X, the temperature tends to become high due to cauterization, whereas the FIG. 5A arrangement hardly includes such region, and so a local temperature rise in the biological tissue can be suppressed. The two circumferentially adjacent electrode portions 22 are respectively brought into contact with one surface and the other surface of the biological tissue, so that the entire biological tissue can be cauterized from both surfaces of the biological tissue. When the electrode portion 22 is in contact with only one surface of the biological tissue, the high-frequency energy from the electrode portion 22 does not sufficiently reach a partial region Y of the surface of the biological tissue opposite to the side where the electrode portion 22 is in contact. On the other hand, since such region hardly exists in the FIG. 5A arrangement, it is possible to prevent a region with insufficient cauterization from occurring in the biological tissue. In this manner, one of the two electrode portions 22 circumferentially adjacent to each other is disposed in the proximal side upright portion 52, and the other is disposed in the distal side upright portion 53, and the electrode portions 22 are alternately brought into contact with both surfaces of the biological tissue, whereby it is possible to evenly cauterize the biological tissue, and prevent the biological tissue from being raised to an excessively high temperature.

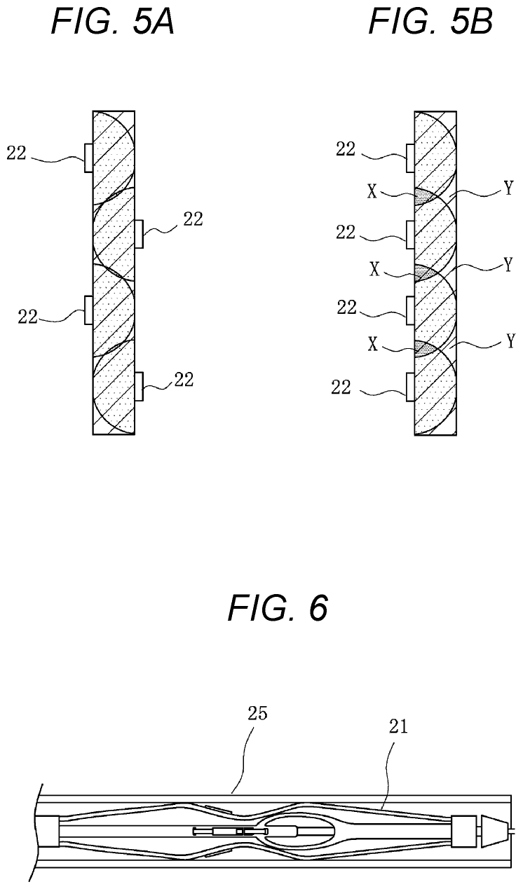

As shown in FIG. 6, the expansion body 21 housed in the storage sheath 25 is in a state of contracting in the radial direction. When the expansion body 21 and the storage sheath 25 move in the axial direction with respect to each other, the expansion body 21 is exposed outward of the storage sheath 25 and expands in the radial direction.

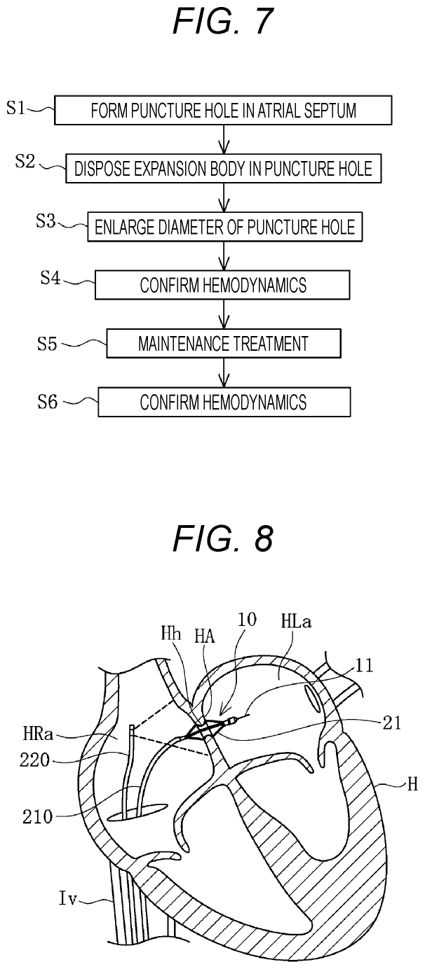

A treatment method using the medical device 10 will be described. The treatment method according to the present embodiment is performed on a patient suffering from a heart failure (left heart failure). More specifically, as shown in FIG. 7, the treatment method is performed on the patient suffering from a chronic heart failure, who has high blood pressure in a left atrium HLa due to myocardial hypertrophy appearing in a left ventricle of the heart H and increased stiffness (hardness).

As shown in FIG. 7, the treatment method according to the present embodiment includes forming the puncture hole Hh in the atrial septum HA (S1), disposing the expansion body 21 in the puncture hole Hh (S2), enlarging the diameter of the puncture hole Hh by using the expansion body 21 (S3), confirming hemodynamics in the vicinity of the puncture hole Hh (S4), performing the maintenance treatment for maintaining the size of the puncture hole Hh (S5), and confirming the hemodynamics in the vicinity of the puncture hole Hh after the maintenance treatment is performed (S6).

When the puncture hole Hh is formed, an operator delivers an introducer 210 in which a guiding sheath and a dilator are combined with each other, to the vicinity of the atrial septum HA. For example, the introducer 210 can be delivered to a right atrium HRa via an inferior vena cava Iv. In addition, the introducer can be delivered using the guide wire 11. The operator can insert the guide wire 11 into the dilator, and can deliver the introducer along the guide wire 11. The introducer and the guide wire 11 can be inserted into a living body by using a known method such as using a blood vessel introducer.

In the forming of the puncture hole Hh in the atrial septum HA (S1), the operator causes a puncture device (not illustrated) to penetrate from the right atrium HRa side toward the left atrium HLa side, thereby forming the puncture hole Hh. For example, a device such as a wire having a sharp distal end can be used as the puncture device. The puncture device is inserted into the dilator, and is delivered to the atrial septum HA. The puncture device can be delivered to the atrial septum HA instead of the guide wire 11 after the guide wire 11 is removed from the dilator.

In the disposing of the expansion body 21 in the puncture hole Hh (S2), the medical device 10 is first delivered to the vicinity of the atrial septum HA along the guide wire 11 inserted in advance. At this time, the distal portion of the medical device 10 penetrates the atrial septum HA, and reaches the left atrium HLa. In addition, when the medical device 10 is inserted, the expansion body 21 is in a state of being stored in the storage sheath 25.

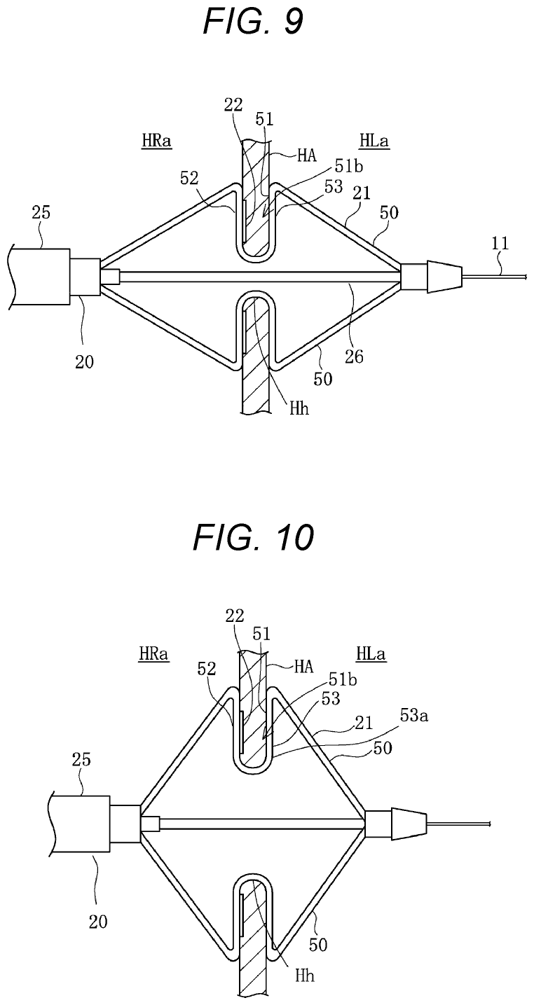

Next, as shown in FIG. 9, the storage sheath 25 is moved to the proximal side so that the expansion body 21 is exposed. In this manner, the outer diameter of the expansion body 21 increases, and the recess portion 51 is arranged in the puncture hole Hh of the atrial septum HA and receives the biological tissue surrounding the puncture hole Hh in the reception space 51b. Due to this, the biological tissue is held between the electrode portion 22 and the distal side upright portion 53 having an opposing surface portion 53a.

To enlarge the outer diameter of the puncture hole Hh by using the expansion body 21 (S3), the operator operates the operation unit 23 in a state where the reception space 51b receives the biological tissue, and the pulling shaft 26 is moved to the proximal side. In this manner, as shown in FIG. 10, the expansion body 21 further expands in the radial direction, and the puncture hole Hh is widened in the radial direction.

After the puncture hole Hh is enlarged, the hemodynamics is confirmed in the vicinity of the puncture hole Hh (S4). As shown in FIG. 8, the operator delivers a hemodynamics confirming device 220 to the right atrium HRa by way of the inferior vena cava Iv. For example, a known echo catheter can be used as the hemodynamics confirming device 220. The operator can display an echo image acquired by the hemodynamics confirming device 220 on a display apparatus such as a display, and can confirm a blood volume passing through the puncture hole Hh, based on a result of the echo image.

Next, the operator performs the maintenance treatment for maintaining the size of the puncture hole Hh (S5). In the maintenance treatment, high-frequency energy is imparted to an edge portion of the puncture hole Hh through the electrode portion 22, thereby cauterizing (heating and cauterizing) the edge portion of the puncture hole Hh by using the high-frequency energy. The high-frequency energy is imparted by applying a voltage between the circumferentially adjacent electrode portions 22. At this time, as described above, since one of the two electrode portions 22 circumferentially adjacent to each other is disposed in the proximal side upright portion 52, and the other is disposed in the distal side upright portion 53, and the electrode portions 22 are alternately brought into contact with both surfaces of the biological tissue, the electrode portions 22 are configured to suppress the temperature rise of the biological tissue and evenly cauterize both surfaces of the biological tissue.

When the biological tissue in the vicinity of the edge portion of the puncture hole Hh is cauterized through the electrode portion 22, a degenerated portion having the degenerated biological tissue is formed in the vicinity of the edge portion. The biological tissue in the degenerated portion is in a state where elasticity is lost. Accordingly, the puncture hole Hh can maintain a shape widened by the expansion body 21.

After the maintenance treatment is performed, the hemodynamics are confirmed again in the vicinity of the puncture hole Hh (S6). In a case where the blood volume passing through the puncture hole Hh reaches a desired volume, the operator decreases the diameter of the expansion body 21. After the expansion body 21 is stored in the storage sheath 25, the expansion body 21 is removed from the puncture hole Hh. Furthermore, the whole medical device 10 is removed outward of the living body, and the treatment is completed.

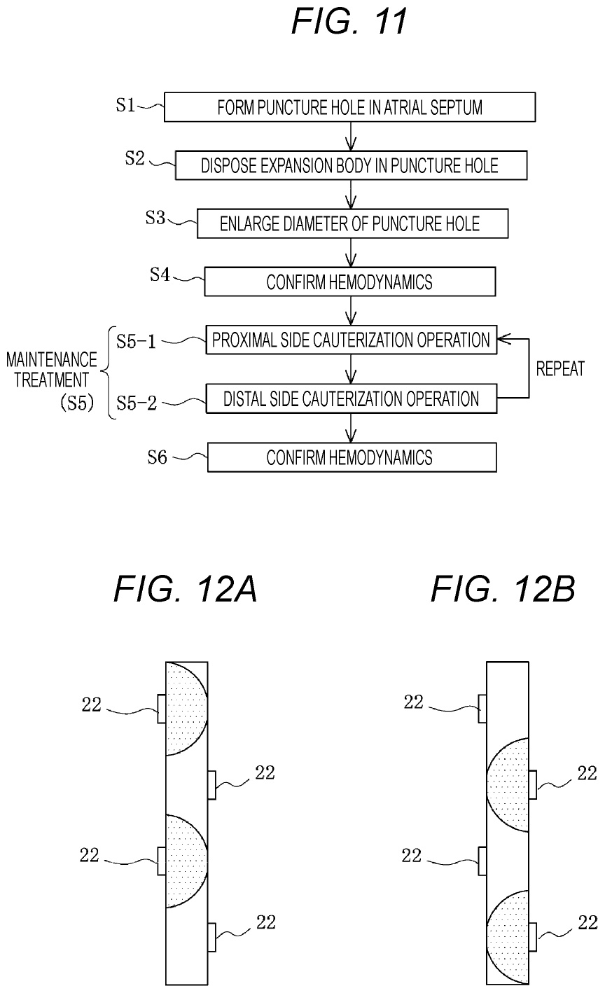

In the performing of the maintenance treatment for maintaining the size of the puncture hole Hh (S5), a voltage may be applied to the electrode portion 22 as follows. As shown in FIG. 11, as the maintenance treatment of S5, the medical device 10 may alternately perform the proximal side cauterization operation (S5-1) and the distal side cauterization operation (S5-2), the proximal side cauterization operation including cauterizing the biological tissue by applying a voltage between the electrode portions arranged in the proximal side upright portion 52 of the electrode portions 22, and the distal side cauterization operation including cauterizing the biological tissue by applying a voltage between the electrode portions 22 arranged in the distal side upright portion 53 of the electrode portions 22. Due to this, as shown in FIGS. 12A and 12B, the regions imparted with energy by the proximal side cauterization operation and the distal side cauterization operation are separated circumferentially, and thus, these regions do not overlap each other. This makes it possible to further suppress the temperature of the biological tissue rising with cauterization.

Next, a modification example of the expansion body will be described. As shown in FIG. 13, an expansion body 60 of the first modification example is formed of a mesh in which a thin wire is knitted. The expansion body 60 includes a recess portion 61 forming a reception space 61a, and a proximal side upright portion 62 and a distal side upright portion 63 are formed in the recess portion 61. One of two electrode portions 64 circumferentially adjacent to each other is disposed in the proximal side upright portion 62, and the other is disposed in the distal side upright portion 63. This allows the electrode portions 64 to be alternately brought into contact, along the circumferential direction, with both surfaces of the biological tissue received in the reception space 61a.

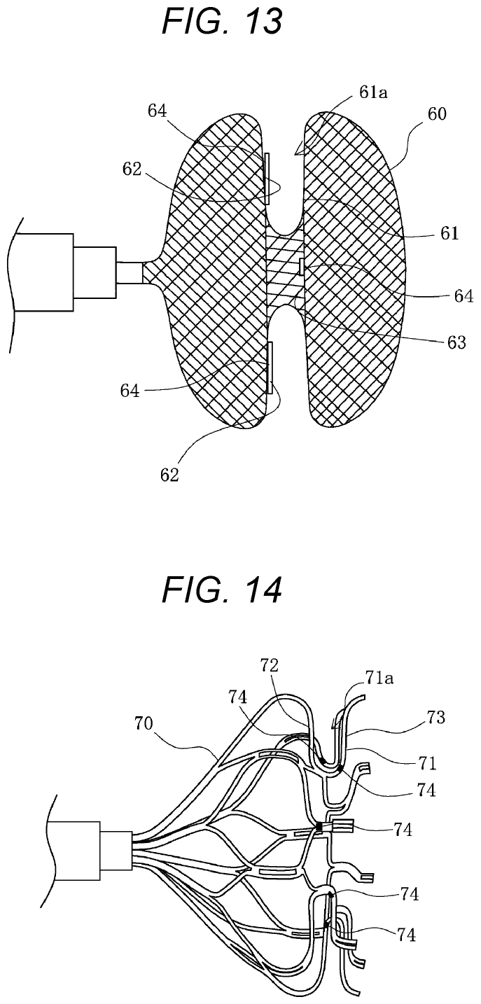

As shown in FIG. 14, an expansion body 70 of the second modification example is formed in a mesh shape in which wires are branched and merged. The expansion body 70 includes a recess portion 71 forming a reception space 71a, and a proximal side upright portion 72 and a distal side upright portion 73 are formed in the recess portion 71. The expansion body 70 does not include a portion on the side distal of the recess portion 71. That is, the distal-most portion of the expansion body 70 is the distal side upright portion 73, and the expansion body 70 does not include structure that is distal of the distal side upright portion 73. That is, the expansion body 70 includes a plurality of wire portions defining the recess portion 71 so as to include four or more recess portions 71 arranged at equal intervals in the circumferential direction of the expansion body 70, and the electrode portions 74 are disposed in the plurality of recess portions 71 in a one-to-one manner. In the present example, the shaft portion does not have a pulling shaft, and the puncture hole Hh can be expanded only by the self-expansion force of the expansion body 70. One of two electrode portions 74 circumferentially adjacent to each other is disposed in the proximal side upright portion 72, and the other is disposed in the distal side upright portion 73. This allows the electrode portions 74 to be alternately brought into contact with both surfaces of the biological tissue received in the reception space 71a.

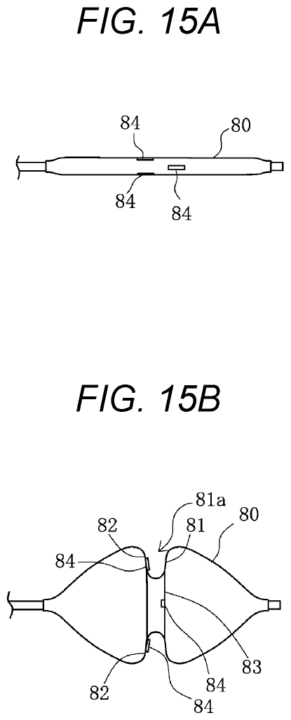

As shown in FIG. 15A, an expansion body 80 of the third modification example is formed of a balloon. A plurality of electrode portions 84 are disposed on a surface of the expansion body 80. As shown in FIG. 15B, when the expansion body 80 is expanded, a recess portion 81 defining a reception space 81a is formed. The recess portion 81 includes a proximal side upright portion 82 and a distal side upright portion 83. In the expansion body 80 in which the recess portion 81 is formed, one of two electrode portions 84 circumferentially adjacent to each other is disposed in the proximal side upright portion 82, and the other is disposed in the distal side upright portion 83. This allows the electrode portions 84 to be alternately brought into contact with both surfaces of the biological tissue received in the reception space 81a.

As described above, the medical device 10 according to the present embodiment includes the expansion body 21 configured to expand and contract in a radial direction; the elongated shaft portion 20 including the distal portion 30 including the proximal end fixing portion 31 to which a proximal end of the expansion body 21 is fixed; the plurality of electrode portions 22 provided along the expansion body 21, in which the expansion body 21 includes the recess portion 51 recessed radially inward when the expansion body 21 expands and defining the reception space 51b configured to receive a biological tissue, the recess portion 51 includes the bottom portion 51a positioned on a radial innermost side, the proximal side upright portion 52 extending radially outward from a proximal end of the bottom portion 51a, and the distal side upright portion 53 extending radially outward from a distal end of the bottom portion 51a, and one of the two electrode portions 22 circumferentially adjacent to each other is arranged in the proximal side upright portion 52, and the other is arranged in the distal side upright portion 53. In the medical device 10 configured in this manner, since the electrode portions 22 are configured to be alternately brought into contact with both surfaces of the biological tissue received in the reception space 51b of the recess portion 51, it is possible to reduce the region where the energy from the circumferentially adjacent electrode portions 22 overlaps, evenly cauterize the biological tissue, and prevent the biological tissue from being raised to an excessively high temperature.

The expansion body 21 may include the plurality of wire portions 50 defining the recess portion 51 to include four or more recess portions 51 arranged at equal intervals in the circumferential direction of the expansion body 21, the plurality of recess portions 51 may each include the bottom portion 51a, the proximal side upright portion 52, and the distal side upright portion 53, and the electrode portions 22 may be disposed in the plurality of recess portions 51 in a one-to-one manner. This allows the electrode portion 22 to more evenly cauterize the biological tissue.

An even number of the electrode portions 22 may be provided in the circumferential direction. This allows all the electrode portions 22 to be alternately arranged with respect to both surfaces of the biological tissue.

A shunt forming method according to the present embodiment is a method of forming a shunt in an atrial septum using the medical device 10 including the expansion body 21 configured to expand and contract in a radial direction, the elongated shaft portion 20 including the distal portion 30 including the proximal end fixing portion 31 to which a proximal end of the expansion body 21 is fixed, and an even number of the electrode portions 22 provided along the expansion body 21, the method in which the expansion body 21 includes the recess portion 51 recessed radially inward when the expansion body 21 expands and defining the reception space 51b configured to receive a biological tissue, the recess portion 51 includes the bottom portion 51a positioned on a radial innermost side, the proximal side upright portion 52 extending radially outward from a proximal end of the bottom portion 51a, and the distal side upright portion 53 extending radially outward from a distal end of the bottom portion 51a, and one of the electrode portions 22 circumferentially adjacent to each other is arranged in the proximal side upright portion 52, and the other of the electrode portions 22 circumferentially adjacent to each other is arranged in the distal side upright portion 53, the recess portion 51 is arranged in a puncture hole formed in an atrial septum to receive a biological tissue surrounding the puncture hole in the reception space 51b defined by the recess portion 51, and to bring the electrode portion 22 into contact with the biological tissue, and the biological tissue is cauterized by applying a voltage to the electrode portion 22 arranged in the proximal side upright portion 52 and the electrode portion 22 arranged in the distal side upright portion 53. In the shunt forming method configured in this manner, since the electrode portions 22 are configured to be alternately brought into contact with both surfaces of the biological tissue, it is possible to reduce the region where the energy from the circumferentially adjacent electrode portions 22 overlaps, evenly cauterize the biological tissue, and prevent the biological tissue from being raised to an excessively high temperature.

When the biological tissue is cauterized, the proximal side cauterization operation and the distal side cauterization operation may be alternately performed, the proximal side cauterization operation including cauterizing the biological tissue by applying a voltage to the electrode portion 22 arranged in the proximal side upright portion 52 of the electrode portion 22, and the distal side cauterization operation including cauterizing the biological tissue by applying a voltage to the electrode portion 22 arranged in the distal side upright portion 53 of the electrode portion 22. Due to this, the proximal side cauterization operation and the distal side cauterization operation are alternately performed, energy is imparted simultaneously from the electrode portions 22 separated by two in the circumferential direction, and the distance between the electrode portions 22 imparted with the voltage can be increased, and therefore the temperature rise of the biological tissue due to the cauterization can be further suppressed.

The detailed description above describes embodiments of a medical device and a shunt forming method representing examples of the new medical device and shunt forming method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A medical device comprising:
an expansion body that is expandable and contractable in a radial direction, the expansion body possessing a proximal end;
an elongated shaft portion including a distal portion, the distal portion of the elongated shaft portion including a proximal end fixing portion to which the proximal end of the expansion body is fixed;
a plurality of electrode portions provided along the expansion body;
the expansion body including a recess portion recessed radially inward when the expansion body expands and defining a reception space configured to receive biological tissue;
the recess portion including a bottom portion positioned radially innermost, a proximal side upright portion extending radially outward from a proximal end of the bottom portion, and a distal side upright portion extending radially outward from a distal end of the bottom portion; and
the plurality of electrode portions including first and second electrode portions positioned circumferentially adjacent to each other, the first electrode portion being arranged at the proximal side upright portion, and the second electrode portion being arranged at the distal side upright portion.

2. The medical device according to claim 1, wherein
the expansion body includes at least four recess portions arranged at equal intervals in a circumferential direction of the expansion body, the expansion body being comprised of a plurality of wire portions that each define one of the recess portions;
the plurality of recess portions each including the bottom portion, the proximal side upright portion, and the distal side upright portion;, and
the electrode portions are disposed on the plurality of recess portions in a one-to-one manner.

3. The medical device according to claim 2, wherein an even number of the electrode portions are provided in a circumferential direction.

4. The medical device according to claim 1, wherein the expansion body possesses a distal end, the distal portion of the elongated shaft portion including a distal end fixing part to which the distal end of the expansion body is fixed.

5. The medical device according to claim 1, wherein the expansion body is a mesh comprised of a knitted wire.

6. The medical device according to claim 1, wherein the expansion body is comprised of wires that branch away from one another and merge towards one another.

7. The medical device according to claim 1, wherein the expansion body is a balloon.

8. A medical device comprising:
an elongated shaft portion including a distal portion;
an expansion body that includes opposite ends and that is fixed to the distal portion of the expansion body, the opposite ends of the expansion body being relatively movable toward and away from one another to cause the expansion body to expand and contract in a radial direction and shift between a contracted state in which the opposite ends of the expandable body are positioned farther away from one another and an expanded state in which the opposite ends of the expandable body are positioned closer to one another;
a storage sheath surrounding the elongated shaft and having an interior, the expansion body being movable relative to the storage sheath so that the expansion body is positionable in the interior of the storage sheath while the expansion body is in the contracted state and is positionable outside the interior of the storage sheath to permit the expansion body to be radially outwardly expanded to the expanded state;
the expansion body including a recess that is recessed radially inward relative to an outer periphery of the expansion body when the expansion body is in the expanded state and that is configured to receive biological tissue, the recess portion including a bottom portion positioned radially innermost;
a plurality of electrodes that are provided on the expansion body and that produce energy applicable to the biological tissue when the biological tissue is received in the recess to cauterize a portion of the biological tissue, the plurality of electrodes including a first electrode and a second electrode;
the first electrode being positioned distal of the second electrode so that when the biological tissue is received in the recess, the first electrode applies the energy to one side of the biological tissue and the second electrode applies the energy to an opposite side of the biological tissue;
the first and second electrodes being positioned radially outwardly of the bottom portion of the recess when the expansion body is in the expanded state and radially inwardly of the outer periphery of the expansion body when the expansion body is in the expanded state; and
all of the electrodes on the expansion body being located at different circumferential positions on the expansion body.

9. The medical device according to claim 8, wherein the expansion body is comprised of a plurality of recesses and a plurality of circumferentially spaced-apart wires, each of the recesses being located on one of the wires.

10. The medical device according to claim 9, wherein the first electrode is mounted on one of the wires and the second electrode is mounted on a different one of the plurality of wires.

11. The medical device according to claim 8, wherein a total number of all of the plurality of electrodes on the expansion body is an even number.

12. The medical device according to claim 8, wherein the expansion body possesses a distal end, the distal portion of the elongated shaft portion including a distal end fixing part to which the distal end of the expansion body is fixed.

13. The medical device according to claim 8, wherein the opposite ends of the expansion body include a distal end and a proximal end, the distal portion of the elongated shaft portion including a proximal end fixing part to which the proximal end of the expansion body is fixed.

14. The medical device according to claim 13, wherein the opposite ends of the expansion body include a distal end and a proximal end, the distal portion of the elongated shaft portion including a distal end fixing part to which the distal end of the expansion body is fixed.

15. The medical device according to claim 8, wherein the expansion body is a mesh comprised of a knitted wire.

16. The medical device according to claim 8, wherein the expansion body is comprised of wires that branch away from one another and merge towards one another.

17. The medical device according to claim 8, wherein the expansion body is a balloon.

18. A method of forming a shunt in an atrial septum using a medical device comprising an expansion body that is expandable and contractable in a radial direction, an elongated shaft portion including a distal portion at which is located a proximal end fixing portion to which a proximal end of the expansion body is fixed, and an even number of electrode portions provided along the expansion body, the electrode portions including first and second electrode portions that are circumferentially adjacent to each other, the expansion body also including a recess portion defining a reception space, the recess portion including a bottom portion positioned at a radial innermost side, a proximal side upright portion extending radially outward from a proximal end of the bottom portion, and a distal side upright portion extending radially outward from a distal end of the bottom portion, the first electrode portion being arranged at the proximal side upright portion and the second electrode portion being arranged at the distal side upright portion; the method comprising:
positioning the recess portion in a puncture hole in the atrial septum so that biological tissue surrounding the puncture hole is positioned in the reception space, and bringing the electrode portions into contact with the biological tissue that is positioned in the reception space; and
applying voltage to the first electrode portion arranged in the proximal side upright portion and the second electrode portion arranged in the distal side upright portion to cauterize the biological tissue in the reception space.

19. The method according to claim 18, wherein when the biological tissue is cauterized, a proximal side cauterization operation and a distal side cauterization operation are alternately performed, the proximal side cauterization operation including cauterizing the biological tissue by applying voltage to the first electrode portion arranged in the proximal side upright portion of the electrode portion, and the distal side cauterization operation including cauterizing the biological tissue by applying voltage to the second electrode portion arranged in the distal side upright portion of the electrode portion.

20. The method according to claim 18, wherein the expansion body includes at least four electrode portions, and the method comprises applying voltage to all of the electrode portions of the expansion body.
